(19) United States
(12) Patent Application Publication
(10) Pub. No.: US 2020/0316131 A1
(43) Pub. Date: Oct. 8, 2020

(54) PHARMACEUTICAL COMPOSITION FOR TREATING DRUG-RESISTANT CANCER COMPRISING EXOSOMES DERIVED FROM DIFFERENTIATING STEM CELLS AS AN ACTIVE INGREDIENT

(71) Applicant: ExoStemTech Co., Ltd., Ansan (KR)

(72) Inventors: Yong Woo CHO, Seongnam (KR); Kyoung-Soo LEE, Suwon (KR); Ji Suk CHOI, Gunpo (KR); Woolee CHO, Busan (KR)

(21) Appl. No.: 16/377,020

(22) Filed: Apr. 5, 2019

(51) Int. Cl. A61K 35/28; A61K 35/35; A61P 35/00; C12N 5/077; C12N 5/0775; C12N 5/079

(52) U.S. Cl. CPC A61K 35/28; A61K 35/35; A61P 35/00; C12N 5/0654; C12N 5/0653; A61K 45/06; C12N 5/0656; C12N 5/0622; C12N 5/0655; C12N 5/0658; C12N 5/0663

(57) ABSTRACT

The present invention relates to a method of treating a subject having drug-resistant cancer, comprising administering a composition comprising exosomes derived from differentiating stem cells as an active ingredient. The exosomes isolated differentiating stem cells according to the present invention have an excellent expression rate of bioactive factors affecting differentiation and have an effect of facilitating reprogramming of cancer stem cells and differentiating them into cancer cells with weakened drug resistance.

### FIELD OF THE INVENTION

The present invention relates to a pharmaceutical composition for treating drug-resistant cancer.

### BACKGROUND OF THE INVENTION

Techniques for culturing stem cells in serum-free medium and extracting cell secretomes or exosomes obtained from the medium have been developed, and components of the extracted material are analyzed by proteomics and genomics techniques. Exosomes are membrane-structured vesicles secreted from many kinds of cells, and they are known to play various roles such as delivering membrane components, proteins, RNA by binding to other cells and tissues, etc.

Conventionally, chemotherapy, radiation therapy, etc. have been used as cancer treatment methods. Chemotherapy is normally a method of inducing the cell proliferation inhibition and death of cancer cells by administering platin, taxol, vinca-derived alkaloid compounds, or chemotherapeutic agents such as 5-fluorouracil, doxorubicin, etc. alone or multiply. However, since most anticancer agents cause severe side effects such as nausea and vomiting, hair loss, discoloration of skin and nails, and nervous system side effects, etc., there is a limitation to the administration dosage and administration period. Radiation therapy is a method of inducing the death of cancer cells by irradiating cancer tissues with high energy radiation. It is common to implement chemotherapy and radiation therapy at the same time for complete recovery of cancer.

As conventional techniques for treating drug-resistant cancer, a method for inhibiting the function of PDGFRα and PDGFRβ, bFGF and/or VEGF-RTK (International Patent PCT/US2006/017922), a method for increasing sensitivity to a cell proliferation inhibitory drug using a casein kinase 2 (CK2) peptide inhibitor (International Patent PCT/CU2007/000010), or a method for targeting cancer stem cells to treat them (International Patent PCT/EP2014/001142), etc. have been disclosed.

Specifically, as techniques for treating drug-resistant cancer, there are techniques of using a chemotherapeutic agent, a casein kinase 2 (CK2) peptide inhibitor, or targeting cancer stem cells.

International Patent PCT/US2006/017922, which is related to a chemotherapeutic agent, confirmed that there was an effect for treating a patient requiring an inhibitor of VEGF-RTK, using an antagonist of receptor tyrosine kinase, particularly, which was a compound acting as an inhibitor of PDGFRα and PDGFRβ, bFGF and/or VEGF-RTK functions.

International Patent PCT/CU2007/000010, which is related to a casein kinase 2 (CK2) peptide inhibitor, provides a CK2 phosphorylation inhibitor (P15 peptide) and a pharmaceutically acceptable cell proliferation inhibitory drug in combination, as the increased activity of CK2 is a factor in increasing resistance of a cell.

International Patent PCT/EP2014/001142, which is related to targeting of cancer stem cells, is a technique related to an antibody recognizing O-acetylated-GD2 ganglioside as a biomarker of cancer stem cell cancer. It was confirmed that such an antibody exhibited the cytotoxic activity of the intrinsic potent to tumor cells, comprising the direct cytotoxicity to cancer stem cells through apoptosis and other apoptosis pathways.

The first conventional technique is to use a compound playing a role of an antagonist of receptor tyrosine kinase, and particularly, it is effective for a patient in need of an inhibitor of VEGF-RTK, but there is a limitation to be applied for various drug-resistant cancer treatment. In addition, there may be a limitation to the administration dosage and administration period as a compound.

The second conventional technique is to use a casein kinase 2 (CK2) peptide inhibitor (P15 peptide) as a composition for treating drug-resistant cancer, and there is a disadvantage in that it is applicable only for cancer stem cells showing CK2 activity, and therefore, there is a limitation to be applied for various drug-resistant cancer treatment.

The third conventional technique is to use an antibody recognizing O-acetylated-GD2 ganglioside as a biomarker of cancer stem cells, and there is a disadvantage in that it is applicable only for cancer stem cells expressing O-acetylated-GD2 ganglioside on the cell surface, and therefore, there is a limitation to be applied for various drug-resistant cancer treatment.

Cancer recurrence has been reported to be caused by cancer stem cells present in cancer tissues. Cancer stem cells are considered to have the similar ability to stem cells, and they have specific surface markers distinct from cancer cells and have self-regeneration and differentiation capabilities. In addition, it has been reported that when cancer stem cells differentiate into specific cells, drug resistance of cancer stem cells is lowered. On the other hand, cancer stem cells are characterized by overexpression of a drug transporter that causes drug resistance. Overexpression of the drug transporter releases drug to the outside of cancer cells, thereby preventing cell death by the drug, leading to cancer recurrence and secondary cancer occurrence. Thus, in order to improve the cancer treatment efficiency fundamentally and reduce recurrence and metastasis of cancer, it is necessary to develop a therapeutic agent which reprograms cancer stem cells into cancer cells with weakened drug resistance or target and kill them.

### SUMMARY OF THE INVENTION

An object of the present invention is to provide a method of treating a subject having drug-resistant cancer, comprising administering a pharmaceutical composition comprising exosomes as an active ingredient, and a method for treating radiation- or drug-resistant cancer by administering the pharmaceutical composition.

In order to achieve the object, the present invention provides a method of treating a subject having drug-resistant cancer, comprising administering a composition comprising exosomes isolated from differentiating stem cells as an active ingredient, and a method for treating radiation- or drug-resistant cancer by administering the pharmaceutical composition.

The present inventors have made intensive efforts to develop a drug-resistant cancer therapeutic agent, and as a result, have found that exosomes derived from differentiating stem cells facilitate reprogramming of cancer stem cells, namely, differentiating them into cancer cells with low drug resistance, and therefore, the cancer treatment efficiency can be maximized, thereby completing the present invention.

Hereinafter, the present invention will be specifically described.

As one aspect, the present invention provides a method of treating a subject having drug-resistant cancer, comprising administering a composition comprising exosomes isolated from differentiating stem cells as an active ingredient.

Herein, the term “stem cell” is a cell which has a characteristic capable of differentiating into various cells by the property of multi potency, when an appropriate signal is given as necessary under the influence of the environment in which the cell is located, in addition to self-replicating ability, and is comprised in fat, bone marrow, cord blood and placenta, etc., and can be utilized for treating various cell damage diseases such as myocardial infarction, cerebral infarction, degenerative arthritis and fracture, and the like.

The stem cells of the present invention may be an autologous or allogeneic stem cells, and may be derived from any type of animals including human and non-human mammals.

Herein, the term “differentiating stem cells” means stem cells which are differentiating into osteocytes or adipocytes, as shown in FIG. 1. From this, exosomes containing genetic information, proteins and growth factors of the osteocytes or adipocytes can be isolated.

Specifically, when stem cells differentiate into osteocytes or adipocytes, the shapes and characteristics of the cells begin to change, and from this time, exosomes are isolated. Therefore, it is different from isolating exosomes from normal undifferentiated stem cells. For example, pre-osteogenic condensation begins to be observed during the differentiation of stem cells into osteocytes; and stem cells differentiating into adipocytes can be confirmed as lipid droplets begin to be formed in the cytoplasm.

The “differentiating stem cells” may be stem cells differentiating into osteocytes or adipocytes, but not limited thereto, and may be stem cells differentiating into osteocytes, myocytes, fibroblasts, astrocytes or nerve cells.

The “differentiating stem cells” may be a marrow-derived stem cells, cord blood-derived stem cells or fat-derived stem cells, but not limited thereto.

The “bone marrow-derived stem cells, cord blood-derived stem cells or fat-derived stem cells” may be human- or animal-derived stem cells, but not limited thereto.

Herein, the term “exosome” is a membrane-structured vesicle of 40˜120 nm in size secreted from various kinds of cells, and it has been known to play various roles of delivering membrane components, proteins, and RNA by binding to other cells and tissues, etc.

The exosome of the present invention may be an exosome isolated from stem cells which are differentiating from bone marrow-derived stem cells, cord blood-derived stem cells or fat-derived stem cells into osteocytes, adipocytes, chondrocytes, myocytes, fibroblasts, astrocytes or nerve cells, but not limited thereto.

The exosomes derived from stem cells which are differentiating into each of various kinds of cells such as osteocytes, adipocytes, chondrocytes, myocytes, fibroblasts, astrocytes or nerve cells may have basic characteristics of stem cells, and may play an important role in anti-tumor immune reaction induction, as containing genes, proteins, growth factors, etc. relative to cell proliferation, differentiation and regeneration of stem cells depending on characteristics of stem cells.

In addition, when they differentiate into each of various kinds of cells such as osteocytes, adipocytes, chondrocytes, myocytes, fibrocytes, astrocytes or nerve cells, etc., they may contain important growth factors, various bioactive proteins and gene information, and the like according to each cell.

In one example of the present invention, it was confirmed that the expression of osteocyte differentiation-related genes such as ALPL (Alkaline phosphatase), BGLAP (Osteocalcin), RUNX2 (Runt-related transcription factor 2), COL1A1 (Collagen, type I, alpha 1), etc. was increased, when exosomes derived from stem cells which were differentiating from human adipose-derived stem cells into osteocytes induced differentiation from CD133+ bone cancer stem cells (CD133+MG63) into osteocytes (FIG. 4 and FIG. 5).

The exosome may be obtained by using exosome isolation methods known in the art, and may be obtained by the isolation method comprising the following steps, but not limited thereto:

1) a step of culturing stem cells in common culture medium and then sub-culturing them in serum-free and antibiotic-free medium;

2) a step of differentiating the stem cells proliferated by sub-culturing into osteocytes, adipocytes, chondrocytes, myocytes, fibroblasts, astrocytes or nerve cells; and
3) a step of isolating and purifying exosomes from stem cells which is differentiating into osteocytes, adipocytes, chondrocytes, myocytes, fibroblasts, astrocytes or nerve cells.

Specifically, the stem cells of the step 1) may be marrow-derived stem cells, cord blood-derived stem cells or fat-derived stem cells, but not limited thereto, and for example, may be fat-derived stem cells.

The fat-derived stem cells may be human or animal-derived stem cells.

As the common culture medium in the step 1), all media for cell culture commonly used in the art can be used, and it may be DMEM (Dulbecco's modified eagle medium) medium, MEM (minimal essential medium) medium, or RPMI 1640 (Rosewell Park Memorial Institute 1640) medium, but not limited thereto.

In addition, one or more kinds of auxiliary components may be added to the cell culture medium if necessary, and as theses auxiliary components, one or more components selected from the group consisting of antibiotics such as penicillin G, streptomycin sulfate and gentamycin, antifungal agents such as amphotericin B and nystatin, and combinations thereof, for preventing contaminant of microorganisms, in addition to fetal bovine, mare or human serum, etc. may be used.

Specifically, it may be DMEM (Dulbecco Modified Eagle Medium high glucose) medium comprising 10% FBS (fetal bovine serum) and penicillin/streptomycin. In addition, it may be DMEM (Dulbecco Modified Eagle Medium high glucose) medium without serum, antibiotics and phenol red, but not limited thereto.

In one example of the present invention, human adipose-derived stem cells (passage 7 or 8) were cultured by using DMEM (Dulbecco Modified Eagle Medium high glucose) medium comprising 10% FBS (fetal bovine serum) and penicillin/streptomycin, and were cultured for 24 hours by replacing it with serum-free, antibiotic-free and phenol red-free medium, before 24 hours prior to isolation of exosomes from the stem cells (Comparative example 1).

In the step 2), “differentiating the stem cells proliferated by sub-culturing into osteocytes, adipocytes, chondrocytes, myocytes, fibroblasts, astrocytes or nerve cells” may be conducted by using a medium composition which induces stem cells to differentiate into osteocytes, adipocytes, chondrocytes, myocytes, fibroblasts, astrocytes or nerve cells.

The medium composition for inducing differentiation may further comprise differentiation-inducing substances such as dexamethasone, insulin, ascorbate, IGF (Insulin-like Growth Factor) and TGF-β1 (Transforming Growth Factor β1, etc., in order to differentiate stem cells into desired cells such as osteocytes, adipocytes, chondrocytes, myocytes, fibroblasts, astrocytes or nerve cells, but not limited thereto.

In one example of the present invention, human adipose-derived stem cells (passage 3 to 7) were cultured by using osteocyte differentiation medium (DMEM high concentration glucose (Dulbecco Modified Eagle Medium) comprising 10% fetal bovine serum, 1% penicillin/streptomycin, 1 μM dexamethasone, 0.5 mM ascorbic acid, and 0.01 M β-glycerophosphate), and were cultured for 24 hours by replacing it with serum-free, antibiotic-free and phenol red-free medium before 24 hours prior to isolation of exosomes from the stem cells which is differentiating into osteocytes (Example 1).

In one example of the present invention, human adipose-derived stem cells (passage 3 to 7) were cultured by using adipocyte differentiation medium (DMEM high concentration glucose (Dulbecco Modified Eagle Medium high glucose) comprising 5% fetal bovine serum, 1 μM dexamethasone, 1 μg/m [symbol] insulin, 100 μM indomethacin, and 0.5 mM 3-isobutyl-1-methylxanthine), and were cultured for 24 hours by replacing it with serum-free, antibiotic-free and phenol red-free medium before 24 hours prior to isolation of exosomes from the stem cells which is differentiating into osteocytes (Example 1).

In the step 3), “isolating and purifying exosomes from stem cells which is differentiating” may finally separate and purify them through centrifugation and filtration of the culture supernatant of the differentiating stem cells.

The centrifugation may be performed at 250 to 500×g for 5 to 10 minutes, or may be performed at 8,000 to 12,000×g for 25 to 45 minutes, and may be performed at 80,000 to 120,000×g for 60 to 80 minutes, but not limited thereto.

The filtration may filter them stepwise using various pore size filters, and may filter them stepwise using a cell strainer having a 4 μm pore size, a filter having a 0.22 μm pore size, and a filter having 500 kDa MWCO (molecular weight cut off), but not limited thereto.

Herein, the term “resistance cancer” is used as the same meaning as “tolerance cancer”, and they are used in combination herein.

Herein, the “tolerance cancer” means cancer which exhibits extremely low sensitivity to anti-cancer agent therapy or radiation therapy, etc., and does not exhibit improvement, alleviation, relief or treatment symptoms of cancer symptoms by the therapy. The tolerance cancer may have tolerance to a specific anti-cancer agent or radiation therapy from the first, and may be generated as the gene in cancer cells is mutated due to long time treatment and no longer shows sensitivity to the same therapeutic agent, although it did not show tolerance at first.

Herein, the tolerance cancer may be all cancer showing tolerance to specifically radiation therapy or anti-cancer agent therapy, but not limited thereto.

The all cancer showing tolerance to radiation therapy or anti-cancer agent therapy may be lung cancer, breast cancer, liver cancer, stomach cancer, colorectal cancer, colon cancer, skin cancer, bladder cancer, pancreatic cancer, prostate cancer, ovarian cancer, cervical cancer, thyroid cancer, renal cancer, fibrosarcoma, melanoma or hematologic cancer, but not limited thereto.

Herein, “drug-resistant cancer” may have resistance, that is, tolerance, to a specific drug, that is, an anti-cancer agent by cancer type, or be caused in case that cancer cells obtain drug tolerance when a specific anti-cancer agent is administered over a long time and the anti-cancer effect cannot be obtained properly, and it may be shown when using anti-cancer drugs usually such as hydrophilic amphiphilic drugs, for example, taxane, vinca alkaloid-based drugs (vinorelbine, vincristine, vinblastine), anthracycline-based drugs (doxorubicin, daunorubicin, epirubicin), epidophyllotoxin (etoposide, teniposide), antimetabolites (methorexate, fluorouracil, cytosar, 5-azacytosine, 6-mercaptopurine, gemcitabine), topotecan, dactinomycin, mitomycin, and the like, as anti-cancer agents to which cancer cells show tolerance known so far.

Herein, the “anti-cancer agent” may be an antimetabolite, an alkylating agent, an anti-tumor antibiotic, a plant alkaloid, an antimitotic drug, a hormonal agent, or a platinum-containing compound, but not limited thereto.

The anti-cancer agent may be antimetabolites such as methotrexate, 6-mercaptopurine, 6-thioguanine, 5-fluorouracil, cytarabine, etc.; alkylating agents such as nitrogen mustard-based compounds (chlorambucil, cyclophosphamide), ethylene imine-based compounds (thiotepa), alkyl sulfonate-based compounds (busulfan), nitreosourea-based compounds (carmustine), triazene-based compounds (dacarbazine), etc.; anti-tumor antibiotics such as actinomycin D, doxorubicin, bleomycin, and mitomycin, plant alkaloids such as vincristine and vinblastine, antimitotic drugs such as toxoid which is an antimitotic drug comprising a taxane ring; or hormonal agents such as adrenocortical hormone and progesterone; platinum-containing compounds such as cisplatin, etc., but not limited thereto.

The pharmaceutical composition for treating drug-resistant cancer according to the present invention is an effective substance for effective treatment of tolerance cancer, and it differs from the prior art in that it uses exosomes isolated and purified from differentiating stem cells.

The exosomes isolated and purified from differentiating stem cells have an excellent expression rate of bioactive factors affecting differentiation and have an effect of lowering drug resistance by efficiently differentiating cancer stem cells or cancer cells.

In other words, stem cell derived-exosomes isolated and purified during the period when stem cells differentiate can facilitate reprogramming of cancer stem cells and differentiate into cancer cells with low drug resistance, and have excellent biocompatibility as they are cell-derived substances, and also have an excellent absorption rate.

Thus, the pharmaceutical composition for treating drug-resistant cancer comprising exosomes derived from differentiating stem cells as an active ingredient according to the present invention can facilitate reprogramming of cancer stem cells and differentiate into cancer cells with low drug resistance, thereby maximizing the efficiency of tolerance cancer treatment, and therefore, it may be usefully used as a therapeutic agent for tolerance cancer.

In one example of the present invention, it was confirmed that when exosomes derived from differentiating stem cells from human adipose-derived stem cells into osteocytes were treated to CD133+ bone cancer stem cells (CD133+MG63), the expression rate of osteocyte differentiation-related genes was increased, that is, the expression rate of drug resistance genes were inhibited in addition to induction of effective differentiation into osteocytes, and it was confirmed that the expression rate of drug transporter genes causing drug resistance was also significantly lowered (FIG. 5 to FIG. 7).

Herein, the exosomes derived from differentiating stem cells according to the present invention which are comprised in the pharmaceutical composition for treating drug-resistant cancer as an active ingredient may be comprised in the pharmaceutical composition at a concentration of 1 to 200 μg/m [symbol] , or at a concentration of 5 to 150 μg/m [symbol] , or at a concentration of 10 to 100 μg/m [symbol] , but not limited thereto, thereby being treated to cancer cells, cancer stem cells or tolerance cancer cells.

Herein, the pharmaceutical composition for treating drug-resistant cancer according to the present invention comprising the exosomes derived from differentiating stem cells as an active ingredient may be used simultaneously, or separately or sequentially with radiation and anti-cancer agents, depending on the situation.

Specifically, the pharmaceutical composition may be administered as an individual therapeutic agent, or may be administered in combination with radiation or other therapeutic agents, and may be administered sequentially or simultaneously with conventional radiation therapy or anti-cancer agents. In addition, it may be administered singly or multiply, and it is important to administer an amount that will achieve the greatest effect in the least amount without side effects, in consideration to all the factors.

The pharmaceutical composition for treating drug-resistant cancer comprises exosomes which contain gene information, proteins and growth factors related to cell differentiation, and thereby, the exosomes can differentiate stem cells into specific cells such as osteocytes, adipocytes, chondrocytes, myocytes, fibroblasts, astrocytes or nerve cells, and in particular, can differentiate cancer stem cells into cancer cells with significantly low drug resistance.

Accordingly, it can be effectively used for overcoming chemical anti-cancer therapy tolerance or overcoming radiation treatment tolerance, and therefore, it is effective to treatment for anti-cancer drug treatment tolerance patients or radiation treatment tolerance patients.

Herein, the pharmaceutical composition for treating drug-resistant cancer may comprise exosomes derived from differentiating stem cells in a pharmaceutically effective amount only, or may comprise one or more of pharmaceutically acceptable carriers, excipients or diluents. The pharmaceutically effective amount means an amount enough to prevent, improve or treat symptoms of drug-resistant cancer.

The pharmaceutically effective amount of exosomes derived from differentiating stem cells according to the present invention may be appropriately modified according to degree of symptoms of drug-resistant cancer, age, body weight, health condition and gender of patients, administration route and treatment period, etc.

In addition, the “pharmaceutically acceptable” means a composition which is physiologically acceptable and does not cause allergic reactions commonly such as gastrointestinal disorders, dizziness, and the like, or reactions similar thereto, when administered to human. Examples of the carrier, excipient and diluent may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate and mineral oils. In addition, fillers, anti-aggregative agents, lubricants, wetting agents, emulsifiers and preservatives, etc. may be further comprised.

The pharmaceutical composition for treating drug-resistant cancer of the present invention may be administered as formulated into a unit dosage form of formulation suitable for administration in the body of patients according to conventional methods in the pharmaceutical arts, and the formulation comprises an effective dose to develop alveoli by one or several administrations. As formulations proper for such a purpose, injection agents such as injectable ampoules, injecting agents such as injection bags, and sprays such as aerosol formulations, etc. are preferable as parenteral administration formulations. The injectable ampoules may be prepared as mixed with injection liquids right before use, and as injection liquids, physiological saline solution, glucose, mannitol, Ringer's solution, etc. may be used. In addition, the injection bags may be made of polyvinyl chloride or polyethylene, and injection bags of Baxter, Becton-Dickinson, Medcep, National Hospital Products or Terumo can be illustrated.

The pharmaceutical formulations may further comprise one or more of pharmaceutically acceptable common inactive carriers, for example, preservatives, pain relief agents, solubilizers or stabilizers, etc. in case of injection agents, and bases, excipients, lubricants or preservatives, etc. in case of formulations for local administration, in addition to the active ingredient.

The composition or pharmaceutical formulations of the present invention prepared in this way may be administered to mammals such as rats, mice, livestock, humans, etc. by various routes such as parenteral, oral and the like, and as the administration method, all methods commonly used in the art may be used. It may be administered by oral, rectal or intravenous, muscular, subcutaneous, intra-uterine or intracerebroventricular injections, etc., but not limited thereto.

Specifically, the administration method may administer exosomes derived from differentiating stem cells by administration intravenously (Intravenous injection) or administration into lung or organ of objects (Topical administration) or inhalation. In addition, the exosomes may be administered using a nebulizer and may be administered using a tube in organ.

As another aspect, the present invention provides a method for treating tolerance cancer comprising a step of administering the pharmaceutical composition for treating drug-resistant cancer according to the present invention comprising exosomes derived from differentiating stem cells in a pharmaceutically effective amount to a subject suffering from radiation or drug-resistant cancer.

Then, the pharmaceutical composition may be administered to a subject independently or the pharmaceutical composition may be administered while performing radiation irradiation, and the pharmaceutical composition may be administered in combination with other compositions for anti-cancer treatment (for example, doxorubicin, vinblastine, etc.).

Herein, the tolerance cancer is as described above.

The term of the present invention “subject” means any animal including human suffering from the tolerance cancer, and the tolerance cancer can be treated by administering the composition of the present invention to the subject.

Herein, the term “treat” means any action that improves or beneficially alters radiation or drug tolerance cancer by administering the pharmaceutical composition of the present invention.

The term of the present invention “administer” means an action that introduces the pharmaceutical composition of the present invention to a subject by any appropriate method, and as the administration route, it may be administered through various oral or parenteral routes as long as it can reach a target tissue.

For the method for treating resistance cancer of the present invention, as the administration route of the pharmaceutical composition, it may be administered through any common route as long as it can reach a target tissue. The pharmaceutical composition of the present invention may be administered by intraperitoneal administration, intravenous administration, intramuscular administration, subcutaneous administration, intradermal administration, oral administration, intranasal administration, intrapulmonary administration, and rectal administration, as desired, but not limited thereto. In addition, the composition may be administered by any equipment in which an active substance can move to a target cell.

Unless otherwise defined, all technical and scientific terms used herein have the same meaning as commonly understood by those skilled in the art to which the present invention belongs. In general, the nomenclature used herein is well known and commonly used in the art.

The exosomes isolated from differentiating stem cells according to the present invention have an excellent expression rate of bioactive factors affecting differentiation and have an effect of facilitating reprogramming of cancer stem cells and differentiating them into cancer cells with weakened drug resistance. In addition, the exosomes derived from differentiating stem cells of the present invention have beneficial advantages when applied as a drug-resistant cancer therapeutic agent, since they are cell-derived substances and have the excellent biocompatibility, and they can minimize side effects of the conventional chemical therapeutic agents and exosomes themselves can play a carrier role, so that supported components can be easily applied to the human body. Accordingly, the pharmaceutical composition for treating drug-resistant cancer according to the present invention can be applied as an agent for treating drug-resistant cancer or a pretreatment composition for treating cancer, using exosomes derived from differentiating stem cells as an active ingredient.

### BRIEF DESCRIPTION OF THE DRAWINGS

FIG. 1 is a mimetic diagram of exosomes derived from human adipose-derived stem cells differentiating into osteocytes or adipocytes according to one example of the present invention and its applications.

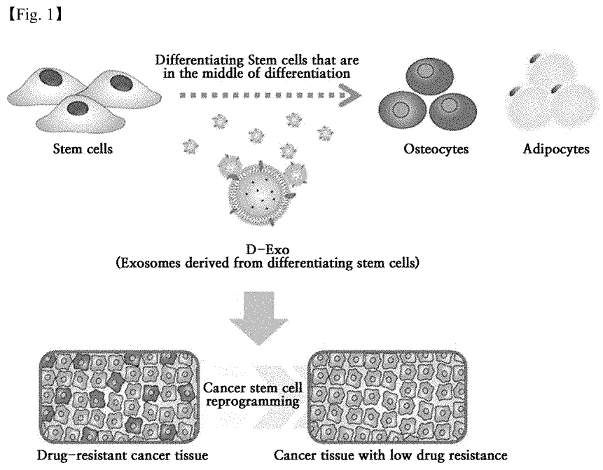

FIG. 2 is a drawing which shows the result of analysis of characteristics of exosomes (D-Exo) isolated from human adipose-derived stem cells differentiating into osteocytes according to one example of the present invention, and is a drawing which shows (A) the structure and shape of the exosomes determined by using a transmission electron microscope, and (B) the size of exosomes determined by using a dynamic light scattering. Herein, exosomes (P-Exo) isolated from human adipose-derived stem cells are used as a control group and the scale bar represents 50 nm.

FIG. 3 is a drawing which shows the result of inducing differentiation of human adipose-derived stem cells into osteocytes by treating exosomes (D-Exo) isolated from stem cells which is differentiating into osteocytes for 21 days by concentration (5 μg/m [symbol] , 10 μg/m [symbol] , 20 μg/m [symbol] , 50 μg/m [symbol] , and 100 μg/m [symbol] ) according to one example of the present invention, and is a drawing which shows (A) the result of Alizarin red s staining, and (B) the calcium detection assay result measuring the absorbance at 562 nm by de-staining the Alizarin red s stained stem cells using 10% cetylpyridinium chloride. Herein, DM is differentiation medium, and P-Exo is the exosome isolated from proliferating human adipose-derived stem cells, and GM is cell culture medium (growth medium).

FIG. 4 is a drawing which shows the result of inducing differentiation of bone cancer stem cells into osteocytes by treating exosomes (D-Exo) isolated from stem cells which is differentiating into osteocytes to CD133+ bone cancer stem cells (CD133+MG63) for 14 days by concentration (5 μg/m [symbol] , 10 μg/m [symbol] , and 30 μg/m [symbol] ) according to one example of the present invention, and is (A) a microscopic observation photograph of bone cancer stem cells after treating exosomes (D-Exo) isolated from stem cells differentiating into osteocytes for 14 days by concentration (5 μg/m [symbol] , 10 μg/m [symbol] , and 30 μg/m [symbol] , and (B) a graph showing the relative change of osteocyte differentiation genes, ALPL (Alkaline phosphatase), BGLAP (Osteocalcin), RUNX2 (Runt-related transcription factor 2) and COL1A1 (Collagen, type I, alpha 1), after treating exosomes (D-Exo) isolated from stem cells differentiating into osteocytes for 14 days by concentration (5 μg/m [symbol] , 10 μg/m [symbol] , and 30 μg/m [symbol] ). Herein, GM is cell culture medium (growth medium), and D-Exo is the exosome isolated from differentiating stem cells from human adipose-derived stem cells into osteocytes.

FIG. 5 is a drawing which shows the expression rate of osteocyte differentiation genes by performing Human Osteogenesis PCR array analysis according to one example of the present invention, and is (A) a graph showing the relative gene expression rate of the group in which the cell culture medium (GM, negative control) is treated and the group in which exosomes (D-Exo) isolated from stem cells which is differentiating into osteocytes are treated, to bone cancer stem cells for 14 days, and (B) a table showing osteocyte differentiation-related genes overexpressed in the group in which exosomes (D-Exo) isolated from stem cells differentiating into osteocytes are treated, compared to the negative control group in which the cell culture medium (GM, growth medium) is treated. Herein, GM is cell culture medium (growth medium), and D-Exo is the exosome isolated from differentiating stem cells from human adipose-derived stem cells into osteocytes.

FIG. 6 is a drawing which shows the expression rate of drug resistance genes using PCR array according to one example of the present invention, and is a drawing which shows (A) the array result showing the relative gene expression rate of the group in which exosomes (D-Exo) isolated from stem cells differentiating into osteocytes for 14 days are treated to bone cancer stem cells, and the group in which the exosomes (D-Exo) are not treated, and (B) a table digitizing the relative gene expression rate.

FIG. 7 is a drawing showing the relative gene expression rate of a drug transporter according to one example of the present invention. Herein, CSC is the group in which exosomes (D-Exo) isolated from differentiating stem cells from bone cancer stem cells into osteocytes are not treated, and CSC+D-Exo is the group in which exosomes (D-Exo) isolated from differentiating stem cells from bone cancer stem cells into osteocytes are treated to bone cancer stem cells for 14 days.

### DETAILED DESCRIPTION OF THE INVENTION

Hereinafter, the configuration and effects of the present invention will be described in more detail through examples. These examples are intended to illustrate the present invention only, but the scope of the present invention is not limited by these examples.

### EXAMPLES

### Example 1: Isolation of Exosomes from Stem Cells Differentiating into Osteocytes or Adipocytes

To isolate each exosome from stem cells differentiating into osteocytes or adipocytes, human adipose-derived stem cells sub-cultured by passages 3 to 7 were cultured in osteocyte differentiation medium (DMEM high concentration glucose (Dulbecco Modified Eagle Medium) comprising 10% fetal bovine serum, 1% penicillin/streptomycin, 1 μM dexamethasone, 0.5 mM ascorbic acid and 0.01 M β-glycerophosphate or adipocyte differentiation medium (DMEM high concentration glucose (Dulbecco's Modified Eagle's Medium high glucose) comprising 5% fetal bovine serum, 1 μM dexamethasone, 1 μg/m [symbol] insulin, 100 μM indomethacin and 0.5 mM 3-isobutyl-1-methylxanthine, respectively, and were maintained for 24 hours by replacing it with DMEM medium that was serum-free and antibiotic-free medium without phenol red for 24 hours once per 3 days. After 24 hours, the culture supernatant of differentiating stem cells was collected to isolate exosomes (D-Exo).

The collected cell culture supernatant was centrifuged at 300×g for 5 minutes to remove cells, and was centrifuged at 10,000×g for 30 minutes to remove cell secretions. Then, it was centrifuged at 100,000×g for 70 minutes using an ultracentrifuge to obtain exosome precipitates. After this, it was washed at 100,000×g for 70 minutes twice in total by adding phosphate-buffered saline (PBS). The washed exosome precipitates were resuspended in phosphate-buffered saline.

### Comparative Example 1: Isolation of Exosomes (P-Exo) from Proliferating Stem Cells

In order to compare with the efficacy of exosomes derived from stem cells differentiating into osteocytes or adipocytes, exosomes (P-Exo) were isolated from proliferating human adipose-derived stem cells as a comparative control group from the supernatant obtained by culturing human adipose-derived stem cells sub-cultured by passages 3 to 7 in common culture medium (DMEM (Dulbecco Modified Eagle Medium) comprising 10% fetal bovine serum and 1% penicillin/streptomycin, and then maintaining them for 24 hours by replacing it with DMEM medium that was serum-free and antibiotic-free medium without phenol red.

The collected cell culture supernatant was centrifuged at 300×g for 5 minutes to remove cells, and was centrifuged at 10,000×g for 30 minutes to remove cell secretions. Then, it was centrifuged at 100,000×g for 70 minutes using an ultracentrifuge to obtain exosome precipitates. After this, it was washed at 100,000×g for 70 minutes twice in total by adding phosphate-buffered saline (PBS). The washed exosome precipitates were resuspended in phosphate-buffered saline.

### Example 2: Analysis of Characteristics of Exosomes (D-Exo) Isolated from Stem Cells Differentiating into Osteocytes

The size and shape of the exosomes derived from Example 1 and Comparative example 1 were confirmed using a transmission electron microscope and a dynamic light scattering.

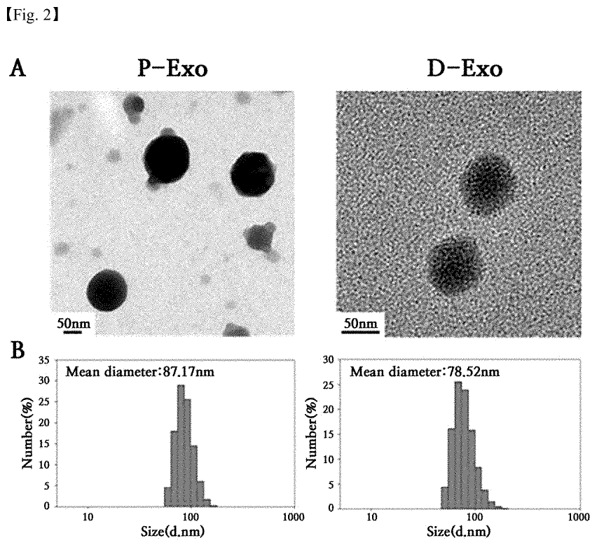

As a result, the shape of each exosome isolated could be confirmed by the transmission electron microscope (A of FIG. 2), and the size of exosomes was confirmed as the size of exosomes (D-Exo) isolated from stem cells differentiating into osteocytes was about 78.52 nm on average, and the size of exosomes (P-Exo) isolated from proliferating stem cells was about 87.17 nm on average (B of FIG. 2).

### Example 3: Osteocyte Differentiation Induction of Human Adipose-Derived Stem Cells Using Exosomes (D-Exo) Isolated from Stem Cells Differentiating into Osteocytes

To confirm the differentiation-inducing ability of exosomes (D-Exo) isolated from stem cells differentiating into osteocytes from human adipose-derived stem cells, a medium composition comprising exosomes derived from stem cells differentiating into osteocytes was used. Here, a medium composition comprising exosomes (P-Exo) isolated from proliferating stem cells was used as a comparative control group. The medium composition was used by adding exosomes (D-Exo) isolated from stem cells differentiating into osteocytes at a concentration of 5 μg/m [symbol] , 10 μg/m [symbol] , 20 μg/m [symbol] , 50 μg/m [symbol] , and 100 μg/m [symbol] to common culture medium (DMEM (Dulbecco Modified Eagle Medium) comprising 10% fetal bovine serum and 1% penicillin/streptomycin). After treating the medium compositions to human adipose-derived stem cells (hASCs), respectively, the medium compositions were replaced once per 3 days for 14 days. Then, as a negative control group (Growth medium, GM), stem cells cultured in DMEM high concentration glucose (Dulbecco's Modified Eagle's Medium high glucose) medium comprising 10% fetal bovine serum and 1% penicillin/streptomycin were used, and as a positive control group (Differentiation medium, DM), stem cells cultured in DMEM high concentration glucose (Dulbecco's Modified Eagle's Medium high glucose) medium comprising 10% fetal bovine serum, 1% penicillin/streptomycin, 1 μM dexamethasone, 0.5 mM ascorbic acid, and 0.01 M β-glycerophosphate were used.

Then, for stem cells in which differentiation into osteocytes was induced for 14 days, Alizarin red s staining was used to analyze whether cells were differentiated. In addition, Alizarin red s stained stem cells were de-stained using 10% cetylpyridinium chloride and the absorbance at 562 nm was measured, thereby quantifying the degree of differentiation.

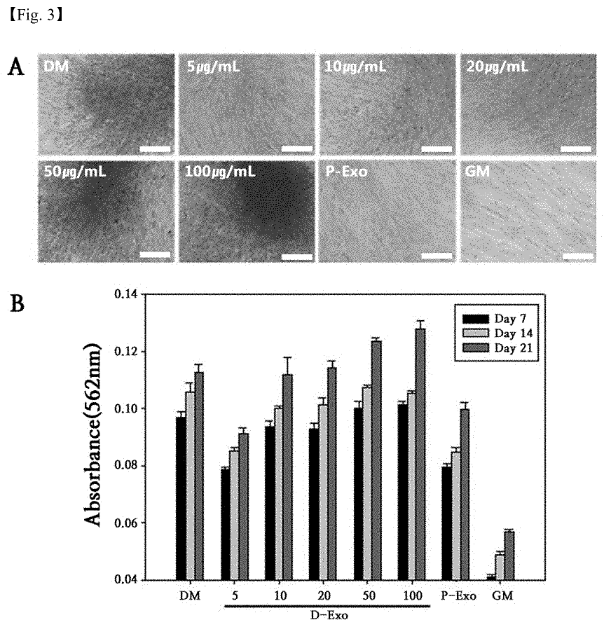

As a result, it could be confirmed that when treating exosomes (D-Exo) isolated from stem cells differentiating into osteocytes at a concentration of 10 μg/m [symbol] or more for 14 days, osteocyte differentiation was induced at a similar level to the positive control group. On the other hand, it was confirmed that in the case of stem cells treated with exosomes (P-Exo) isolated from proliferating stem cells, they did not differentiate into osteocytes, but only proliferation was achieved (FIG. 3).

### Example 4: Bone Cancer Cell Differentiation Induction of Bone Cancer Stem Cells Using Exosomes (D-Exo) Isolated from Stem Cells Differentiating into Osteocytes and Analysis of Expression Rate of Osteocyte Differentiation Genes Using PCR Array

To confirm the differentiation-inducing ability of exosomes (D-Exo) isolated from stem cells differentiating into osteocytes from human adipose-derived stem cells, a medium composition comprising exosomes derived from stem cells differentiating into osteocytes were used. The medium composition was used by adding exosomes (D-Exo) isolated from stem cells differentiating into osteocytes at a concentration of 5 μg/m [symbol] , 10 μg/m [symbol] , and 30 μg/m [symbol] to common culture medium (DMEM (Dulbecco Modified Eagle Medium) comprising 10% fetal bovine serum and 1% penicillin/streptomycin). The medium composition was treated to cultured CD133+ bone cancer stem cells (CD133+MG63), respectively, and then the medium composition was replaced once per 3 days for 14 days. Then, as a negative control group (Growth medium, GM), stem cells cultured in DMEM high concentration glucose (Dulbecco's Modified Eagle's Medium high glucose) comprising 3% fetal bovine serum and 1% penicillin/streptomycin were used.

After this, for bone cancer stem cells in which differentiation into osteocytes was induced for 14 days, whether cells were differentiated was analyzed using a PCR analysis technique.

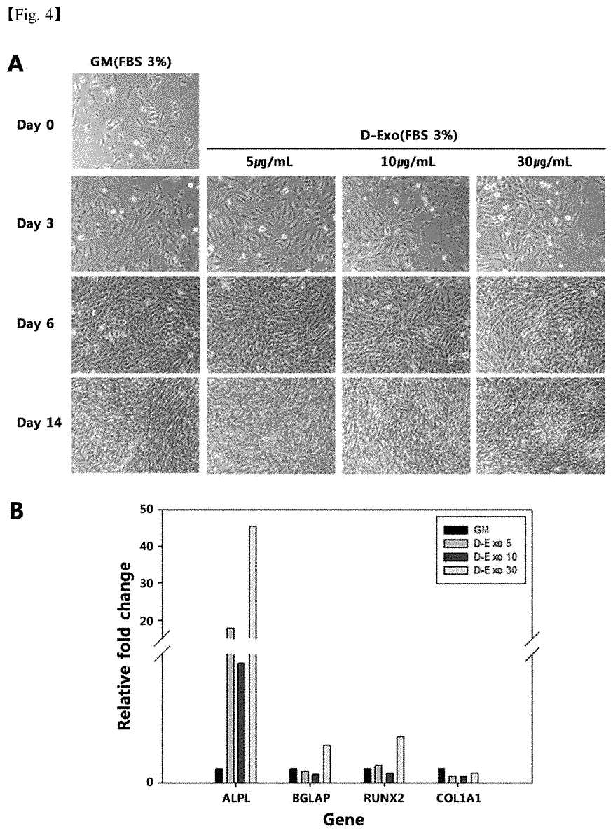

As a result, it was confirmed that when treating exosomes (D-Exo) isolated from stem cells differentiating into osteocytes at a concentration of 30 μg/m [symbol] or more for 14 days, osteocyte differentiation genes such as ALPL (Alkaline phosphatase), BGLAP (Osteocalcin), RUNX2 (Runt-related transcription factor 2), etc. were expressed 2 to 45 times higher than the negative control group (FIG. 4).

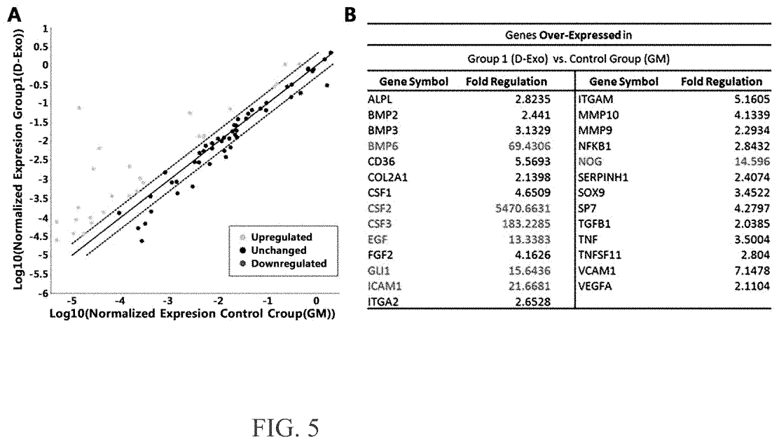

In addition, as the result of qualifying the mRNA expression rate through Human Osteogenesis PCR array analysis, it was confirmed that 27 genes of 73 kinds of osteocyte differentiation-related genes in total were expressed over 2 times higher in the group in which exosomes (D-Exo) isolated from stem cells differentiating into osteocytes were treated (FIG. 5).

### Example 5: Analysis of Expression Rate of Drug Resistance Gene Using PCR Array and Analysis of Relative Gene Expression Rate of Drug Transporter

To confirm the bone cancer stem cell drug resistance inhibitory ability of exosomes (D-Exo) isolated from stem cells differentiating into osteocytes from human adipose-derived stem cells, a medium composition comprising exosomes (D-Exo) isolated from stem cells which were differentiating into osteocytes was used. The medium composition was used by adding exosomes derived from stem cells differentiating into osteocytes at a concentration of 30 μg/m [symbol] to common culture medium (DMEM (Dulbecco Modified Eagle Medium) comprising 10% fetal bovine serum and 1% penicillin/streptomycin). The medium composition was treated to the cultured CD133+ bone cancer stem cells (CD133+MG63), and then the medium composition was replaced once per 3 days for 14 days. Then, as a negative control group (Growth medium, GM), stem cells cultured in DMEM high concentration glucose (Dulbecco's Modified Eagle's Medium high glucose) medium comprising 3% fetal bovine serum and 1% penicillin/streptomycin were used.

After this, for bone cancer stem cells in which differentiation into osteocytes was induced for 14 days, whether cells were differentiated was analyzed using a PCR analysis technique.

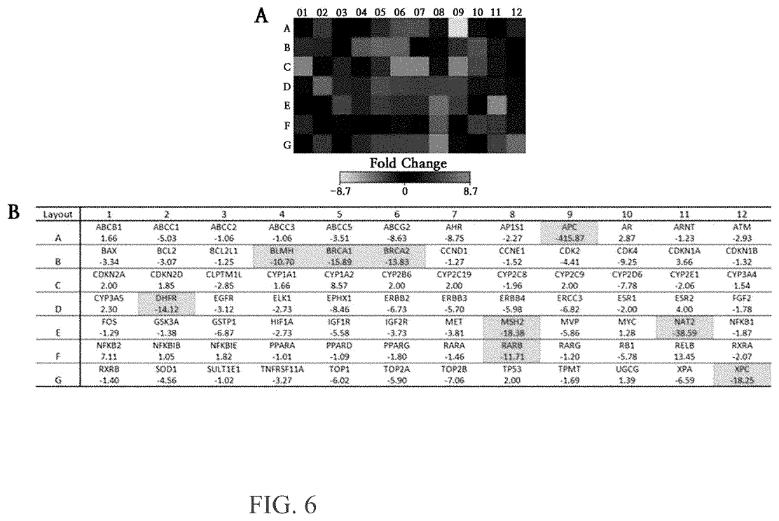

As a result, it was confirmed that 64 genes among 84 kinds of drug resistance-related genes in total were inhibited in the group in which exosomes (D-Exo) isolated from stem cells differentiating into osteocytes were treated (A of FIG. 6). In particular, it was confirmed that APC, BLMH, BRCA1, BRCA2, DHFR, MSH2, NAT2, RARB, and XPC genes were inhibited 10 to 415 times (B of FIG. 6).

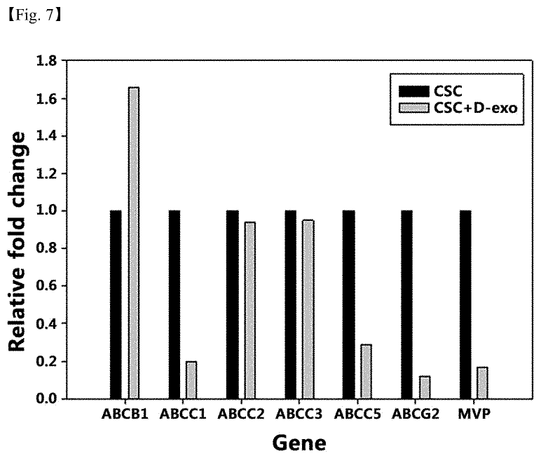

In addition, the expression of ABCC1, ABCC5, ABCG2, and MVP genes among drug transporter genes which directly affected drug resistance was significantly lowered (FIG. 7).

What is claimed is:

1. A method of treating a subject having drug-resistant cancer, comprising administering a composition comprising exosomes isolated from differentiating stem cells as an active ingredient.

2. The method according to claim 1, wherein the differentiating stem cells are stem cells differentiating into osteocytes, adipocytes, cartilage cells, myocytes, fibroblasts, astrocytes or nerve cells.

3. The method according to claim 2, wherein the stem cells are marrow-derived stem cells, cord blood-derived stem cells or fat-derived stem cells.

4. The method according to claim 3, wherein the marrow-derived stem cells, cord blood-derived stem cells or fat-derived stem cells are human- or animal-derived stem cells.

5. The method according to claim 1, wherein the composition comprises exosomes at a concentration of 1 to 200 μg/m [symbol] .

6. The method according to claim 1, wherein the composition is administered simultaneously, separately or sequentially with radiation or an anticancer agent.

7. The method according to claim 6, wherein the anticancer agent is an antimetabolite, an alkylating agent, an anti-tumor antibiotic, a plant alkaloid, an antimitotic drug, a hormonal agent, or a platinum-containing compound.

8. The method according to claim 1, wherein the drug-resistant cancer is lung cancer, breast cancer, liver cancer, stomach cancer, colorectal cancer, colon cancer, skin cancer, bladder cancer, pancreatic cancer, prostate cancer, ovarian cancer, cervical cancer, thyroid cancer, renal cancer, fibrosarcoma, melanoma or hematologic cancer.
